(19) United States
(12) Patent Application Publication
(10) Pub. No.: US 2016/0029878 A1
(43) Pub. Date: Feb. 4, 2016

(54) ENDOSCOPE, PART FIXING STRUCTURE FOR ENDOSCOPE, AND PART FIXING METHOD FOR ENDOSCOPE

(71) Applicant: FUJIFILM Corporation, Tokyo (JP)

(72) Inventors: Masayuki YAMAZAKI, Kanagawa (JP); Kenji YAMANE, Kanagawa (JP); Tomoya SUZUKI, Kanagawa (JP)

(21) Appl. No.: 14/801,806

(22) Filed: Jul. 16, 2015

(30) Foreign Application Priority Data
Aug. 1, 2014 (JP) 2014-158109

(51) Int. Cl. A61B 1/01; B23K 26/21; A61B 1/008; A61B 1/005; A61B 1/00

(52) U.S. Cl. CPC A61B 1/01; A61B 1/0057; A61B 1/0055; A61B 1/0011; A61B 1/008; B23K 26/21

(57) ABSTRACT

An endoscope includes an insertion part in which a rigid distal end part, a bending part, and a flexible tube part are provided; an operating part having a bending operation mechanism; an operating wire; a wire guide pipe; and a sleeve of which the inside is formed in a hollow tubular shape along an axial direction parallel to a longitudinal axis of the insertion part and which is externally fitted to and hold a distal end part of the operating wire or the wire guide pipe. The sleeve includes a slit that is formed by cutting out a partition wall of the insertion part. An opening width of the slit in a circumferential direction perpendicular to the axial direction is made to be smaller than an external diameter of the distal end part of the operating wire inserted through the sleeve or the wire guide pipe.

### CROSS-REFERENCE TO RELATED APPLICATIONS

The present application claims priority under 35 U.S.C. §119 to Japanese Patent Application No. 2014-158109, filed on Aug. 1, 2014. Each of the above application(s) is hereby expressly incorporated by reference, in its entirety, into the present application.

### BACKGROUND

1. Field of the Disclosure

The present disclosure relates to an endoscope, a part fixing structure for an endoscope, and a part fixing method for an endoscope, and particularly, to a fixing structure that is arranged inside an insertion part of an endoscope, and fixes a distal end part of an operating wire, which bends a bending part, and a wire guide pipe through which the operating wire is inserted.

2. Description of the Related Art

Generally, flexible endoscopes that are widely used for medical treatment, include an insertion part that is inserted into the body of a subject, and an operating part that is gripped and operated by a surgeon. The insertion part is configured such that a rigid distal end part into which an objective optical system, a solid-state image pick-up element, or the like is built, a bending part that is bent by the bending operation of the operating part, and an elongated flexible tube part having flexibility are coupled together sequentially from a distal end side.

The bending part is configured such that a plurality of nodal rings are rotatably coupled together in order to be made bendable, the distal end part of the operating wire passing through the insertion part is fixed to a leading nodal ring or the rigid distal end part, and a base end of the operating wire is connected to a bending operation mechanism of the operating part.

An angle knob operated by a surgeon is provided as a constituent element of the bending operation mechanism in the operating part, and the operation of the angle knob causes the operating wire to be pushed and pulled and causes the bending part to be bent in an upward-downward direction or a leftward-rightward direction. Accordingly, the orientation of the rigid distal end part can be changed by the operation of the angle knob by the surgeon.

Additionally, it is generally known that the operating wire is inserted through and arranged in the wire guide pipe in order to prevent damage caused by friction or the like with other built-in elements of the insertion part, and the distal end part of the wire guide pipe is fixed to a rear nodal ring or a distal end of the flexible tube part.

As the structure in which the distal end part of such an operating wire or the wire guide pipe is fixed to the ring member that constitutes a portion of the outer peripheral part of the insertion part, the following are disclosed in JP1997-75300A (JP-H09-75300A), JP2014-23919A, and JP2013-223655A.

According to JP1997-75300A (JP-H09-75300A), a pipe-like locking part is formed to protrude from an inner peripheral surface of a ring member (a head coupling, an angle coupling), and a distal end part of an operating wire (angle wire) or a wire guide pipe is inserted through a hole in the locking part. Then, the operating wire is locked to the ring member by a terminal metal fitting anchored to a distal end of the operating wire being locked to the locking part with respect to the pulling of the operating wire. Additionally, when the distal end part of the wire guide pipe is engaged with a hole in the locking part subjected to tapping, the distal end part of the wire guide pipe is fixed to the ring member.

A fixing structure of a distal end part of an operating wire is disclosed in JP2014-23919A, and a pair of protruding parts protruding from an inner peripheral surface of a ring member (distal end ring) are provided (refer to FIG. 9 of JP2014-23919A). Then, the distal end part of the operating wire is fixed to the ring member by pressing and plastically deforming the distal end part of the operating wire so as to sandwich the distal end part between the protruding parts.

A fixing structure of a distal end part of a wire guide pipe (guide pipe) is disclosed in JP2013-223655A, and a pair of bending pieces that face each other in a circumferential direction are provided by forming a slit in a ring member (tubular mouthpiece) and by being bent radially inward. Then, a distal end part of the wire guide pipe is fixed to the ring member by sandwiching and welding the distal end part of the wire guide pipe between the pair of the bending pieces.

### SUMMARY

However, if the locking part having the hole through which the distal end part of the operating wire or the wire guide pipe is inserted is formed so as to protrude to an inner peripheral surface side of the ring member as in JP1997-75300A (JP-H09-75300A), a portion of the locking part protrudes farther inward than at least the position of the hole of the locking part through which the operating wire or the wire guide pipe is inserted. Therefore, there is a problem in that an internal space in the insertion part becomes small.

In JP2014-23919A, the pair of protruding parts that protrude radially inward from the inner peripheral surface of the ring member protrude farther inward than the operating wire, and there is a problem in that an internal space in the insertion part becomes small.

Additionally, a groove that sandwiches the distal end part of the operating wire between the pair of protruding parts is formed with a given width and the groove itself does not have a portion that prevents the operating wire from coming off the groove. Therefore, there is a drawback in that the operating wire easily comes off from the groove.

Since the pair of bending pieces of the ring member in JP2013-223655A sandwich the distal end part of the wire guide pipe with their distal end parts, the bending pieces do not protrude radially inward farther than the wire guide pipe, and the internal space in the insertion part can be widened. However, since the distal end parts of the pair of bending pieces abut against other symmetrical positions on the outer peripheral surface of the wire guide pipe (positions opposite to each other by 180 degrees in the circumferential direction), there is a drawback that the wire guide pipe easily comes off between the bending pieces.

The disclosure has been made in view of such circumstances to provide an endoscope, a part fixing structure for an endoscope, and a part fixing method for an endoscope that can reliably fix an insertion member, such as an operating wire or a wire guide pipe, which is inserted through the insertion part of the endoscope, and can widen an internal space in an insertion part.

In order to achieve the object, according to an aspect of the disclosure, there is provided an endoscope including: an insertion part in which a rigid distal end part, a bending part, and a flexible tube part are provided in order from a distal end side to a base end side; an operating part having a bending operation mechanism that performs bending of the bending part, the operating part being provided on the base end side of the insertion part; an operating wire having a distal end part and a base end part, the distal end part being fixed to a distal end part of the bending part or the rigid distal end part, the base end part being connected to the bending operation mechanism, the operating wire being configured to bend the bending part by the bending operation mechanism being operated; a wire guide pipe through which the operating wire is inserted, having a distal end part and a base end part, the distal end part being fixed to a base end part of the bending part or a distal end part of the flexible tube part, the base end part being fixed to an inside of the operating part or a base end part of the flexible tube part; and a sleeve of which the inside is formed in a hollow tubular shape along an axial direction parallel to a longitudinal axis of the insertion part and which is externally fitted to and hold the distal end part of the operating wire or the wire guide pipe, wherein the sleeve includes a slit that is formed by cutting out a partition wall of the insertion part on a center side along the axial direction in a section perpendicular to the axial direction, and allows the inside and the outside of the sleeve to communicate with each other, wherein the slit maintains a maximum internal diameter of the inside the hollow tubular shape, and wherein an opening width of the slit in a circumferential direction perpendicular to the axial direction is made to be smaller than an external diameter of a distal end part of the operating wire inserted through the sleeve or the wire guide pipe.

According to the above aspect, in the sleeve that holds the distal end part of the operating wire or the wire guide pipe, the slit is formed in the partition wall of the insertion part on the center side. Accordingly, the amount of protrusion of the sleeve to the center side of the insertion part can be made small, and an internal space in the insertion part can be enlarged. Additionally, since the opening width of the slit is made to be smaller than the external diameter of the distal end part of the operating wire or the wire guide pipe, the distal end part of the operating wire or the wire guide pipe does not easily come off the sleeve, and the distal end part of the operating wire or the wire guide pipe can be reliably fixed.

In the endoscope according to another aspect of the disclosure, the sleeve may have a laser welded part including a cutout part formed by partially cutting out an outer wall defining the slit in a radial direction perpendicular to the axial direction.

According to the above aspect, laser welding between the sleeve and the operating wire or the wire guide pipe is performed in the laser welded part. Accordingly, even if the size of a welding portion expands, the expanded portion is prevented from protruding farther than a peripheral portion.

The endoscope according to another aspect of the disclosure may further include a ring member that constitutes a part of an outer peripheral part of the insertion part, wherein the sleeve is constituted separately from the ring member, and is fixed to the inside of the ring member

According to the above aspect, the formation of the ring member becomes easy, and it is possible to adopt a method of fixing the distal end part of the operating wire or the wire guide pipe to the sleeve and then fixing the sleeve to the ring member in a state where the sleeve is not fixed to the ring member, during the work in which the distal end part of the operating wire or the wire guide pipe is fixed to the ring member. Therefore, the fixing work becomes easy.

In the endoscope according to another aspect of the disclosure, the sleeve may be fixed to the inside of the ring member by laser welding.

In the endoscope according to another aspect of the disclosure, either one of the sleeve and the ring member may have a fixed part, and the other one of the sleeve and the ring member may have a fixing part that is engageable with the fixed part. The sleeve may be fixed in a state where the sleeve is aligned with the ring member by engaging the fixed part with the fixing part.

According to the above aspect, the sleeve can be reliably fixed to the predetermined position of the ring member, coming-off of the sleeve from the ring member can be prevented, and variations between products with respect to the fixed position of the sleeve can be eliminated.

In the endoscope according to another aspect of the disclosure, the fixing part may be constituted by a projection provided on a surface of the sleeve opposite to the slit. The fixed part may be constituted by a hole or a groove that is provided in the ring member and has a shape corresponding to the projection. The sleeve may be fixed in a state where the sleeve is aligned with the ring member by fitting the projection into the hole or the groove.

In the endoscope according to another aspect of the disclosure, the sleeve may have a position regulating part that abuts against an inner surface of the ring member when the projection is fitted into the hole or the groove.

According to the above aspect, the sleeve can be reliably fixed to the predetermined position of the ring member, and shaking of the sleeve can be prevented.

According to another aspect of the disclosure, there is provided a part fixing structure for fixing an insertion member, which is inserted through an insertion part of an endoscope including: a sleeve of which the inside is formed in a hollow tubular shape along an axial direction parallel to a longitudinal axis of the insertion part and which is externally fitted to and hold a part of the insertion member, wherein the sleeve includes a slit that is formed by cutting out a partition wall of the insertion part on a center side along the axial direction in a section perpendicular to the axial direction, and allows the inside and the outside of the sleeve to communicate with each other, wherein the slit maintains a maximum internal diameter of the inside the hollow tubular shape, and wherein an opening width of the slit in a direction perpendicular to the axial direction is made to be smaller than an external diameter of the insertion member.

According to the above aspect, in the tubular sleeve to which a portion of the insertion member inserted through the insertion part of the endoscope is externally fitted and fixed, the slit is forming in the partition wall of the insertion part on the center side. Accordingly, the amount of protrusion of the sleeve to the center side of the insertion part can be made small, and the internal space in the insertion part can be enlarged. Additionally, since the opening width of the slit is smaller than the external diameter of the insertion member, the insertion member does not easily come off from the sleeve, and the insertion member can be reliably fixed.

According to still another aspect of the disclosure, there is provided a part fixing method for fixing an insertion member inserted through an insertion part of an endoscope, including: a step of having a sleeve to be externally fitted to a part of the insertion member, of which sleeve the inside is formed in a hollow tubular shape along an axial direction parallel to a longitudinal axis of the insertion part and which includes a slit which is formed by cutting out a partition wall of the insertion part on a center side along the axial direction in a section perpendicular to the axial direction, the slit allowing the inside and the outside of the sleeve to communicate with each other, wherein the slit maintains a maximum internal diameter of the inside the hollow tubular shape, and the opening width of the slit in a direction perpendicular to the axial direction is made to be smaller than the external diameter of the insertion member; and a step of fixing the insertion member to the sleeve by performing laser welding via the slit.

According to the present disclosure, the insertion member, such as the operating wire or the wire guide pipe, which is inserted through the insertion part of the endoscope, can be reliably fixed, and the internal space in the insertion part can be widened.

### BRIEF DESCRIPTION OF THE DRAWINGS

FIG. 1 is a view illustrating the appearance of of an endoscope to which the disclosure is applied.

FIG. 2 is a longitudinal sectional view of the periphery of a coupling portion between a bending part and a flexible tube part that is cut along a longitudinal axis of an insertion part.

FIG. 3 is a longitudinal sectional view of the periphery of a coupling portion between the bending part and a rigid distal end part that is cut along the longitudinal axis of the insertion part.

FIG. 4 is a view conceptually illustrating the bending part and a bending operation mechanism.

FIG. 5 is a perspective view illustrating only a distal end ring, a base end ring, operating wires, and wire guide pipes in the bending part.

FIG. 6 is a cross-sectional view of a sleeve that is provided at an inner upper part of the base end ring and that is cut in a plane orthogonal to the longitudinal axis of the insertion part.

FIG. 7 is a view illustrating the sleeve and the wire guide pipe from a radial outer side of the insertion part.

FIG. 8 is a view illustrating the sleeve and the wire guide pipe from a radial inner side of the insertion part.

FIG. 9 is a view illustrating another embodiment of a configuration for fixing the sleeve to an outer peripheral part of the base end ring, and is a cross-sectional view of the sleeve that is provided at the inner upper part of the base end ring and that is cut in the plane orthogonal to the longitudinal axis of the insertion part.

FIG. 10 is a view illustrating still another embodiment of the configuration for fixing the sleeve to the outer peripheral part of the base end ring, and is a cross-sectional view of the sleeve that is provided at the inner upper part of the base end ring and that is cut in the plane orthogonal to the longitudinal axis of the insertion part.

### DESCRIPTION OF THE EMBODIMENTS

Embodiments of the disclosure will be described below in detail according to the accompanying drawings.

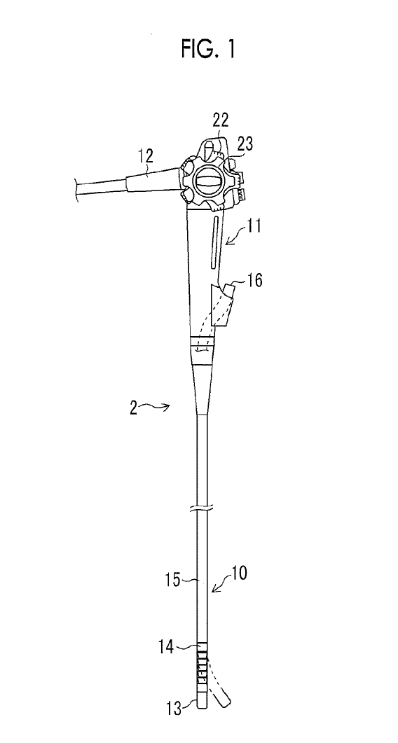

FIG. 1 is a view illustrating the appearance of of an endoscope to which the disclosure is applied. In FIG. 1, an endoscope 2 includes an insertion part 10 that is inserted into a human body, an operating part 11 that is provided continuously with a base end part of the insertion part 10, and a universal cord 12 that is provided continuously with the operating part 11. The universal cord 12 connects the endoscope 2 with a processor device (not illustrated) that processes image signals obtained by image pick-up using the endoscope 2 and a light source device (not illustrated) that emits illumination light guided to the endoscope 2, and has signal cables for transmitting image signals and control signals, a light guide for guiding illumination light, or the like disposed therein.

The insertion part 10 has a rigid distal end part 13, a bending part 14 that is provided continuously with a base end of the rigid distal end part 13, and a flexible tube part 15 that is provided continuously with a base end of the bending part 14.

An image pick-up device for imaging a part to be observed within a body cavity, an illumination light emitting part that radiates illumination light to a part to be observed, or the like is mounted on the rigid distal end part 13.

The flexible tube part 15 has flexibility, and has a length of several meters in order to make the rigid distal end part 13 reach a target position within the human body.

As a surgeon operates an upward-downward angle knob 22 and a leftward-rightward angle knob 23 in a bending operation mechanism provided in the operating part 11, the bending part 14 is bent upward and downward and leftward and rightward, and makes the orientation of the rigid distal end part 13 variable.

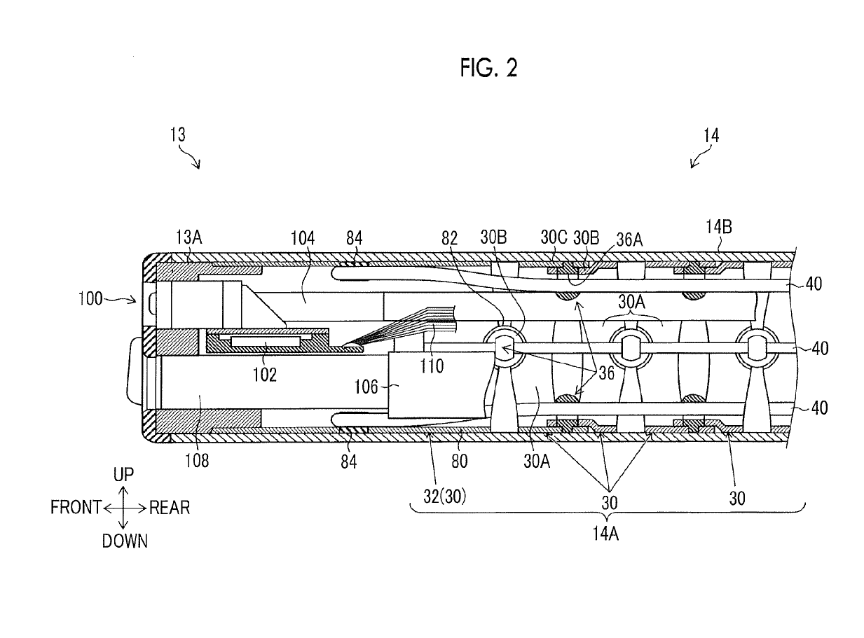

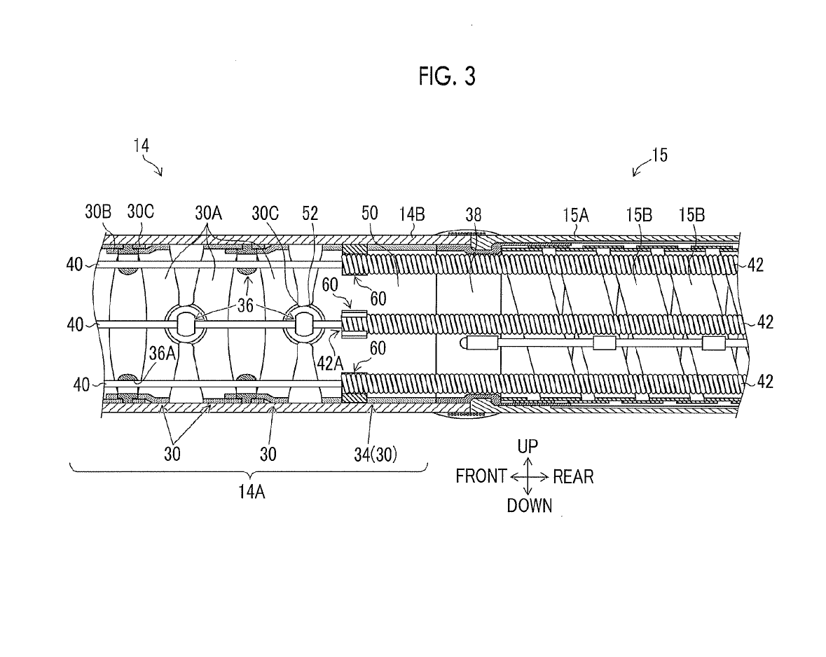

The structure of the bending part 14 is illustrated in FIGS. 2 and 3. FIG. 2 illustrates a longitudinal sectional view of the periphery of the coupling portion between the bending part 14 and the rigid distal end part 13 that is cut along a longitudinal axis (axial center) of the insertion part. FIG. 3 illustrates a longitudinal sectional view of the periphery of the coupling portion between the bending part 14 and the flexible tube part 15 that is cut along a longitudinal axis (axial center) of the insertion part.

As illustrated in these drawings, the bending part 14 has a nodal ring group 14A consisting of a plurality of nodal rings 30 formed of metallic materials, such as stainless steel, and an outer peripheral side of the nodal ring group 14A is configured so as to be covered with an outer peripheral wall 14B constituted of a net-like pipe and a sheath.

The respective nodal rings 30 of the nodal ring group 14A are coupled in series, and each of the nodal rings 30, excluding a distal end ring 32 (refer to FIG. 2) that is a leading nodal ring 30 among the nodal rings 30, and a base end ring 34 (refer to FIG. 3) that is a trailing nodal ring 30, has a ring-shaped outer peripheral part 30A, a pair of tongue pieces 30B and 30B formed to protrude to a distal end side with respect to the outer peripheral part 30A, and a pair of tongue pieces 30C and 30C formed to protrude to a base end side.

The tongue pieces 30B and 30B on the distal end side and the tongue pieces 30C and 30C on the base end side are arranged at different positions which are left and right positions and upper and lower positions in a circumferential direction in the respective nodal rings 30, the tongue pieces 30C and 30C on the base end side of a nodal ring 30 in which the tongue pieces 30B and 30B on the distal end side are arranged at the left and right positions are arranged at the left and right positions, and the tongue pieces 30C and 30C on the base end side of a nodal ring 30 in which the tongue pieces 30B and 30B on the distal end side are arranged at the upper and lower positions are arranged at the upper and lower positions.

In two nodal rings 30 adjacent to each other, the tongue pieces 30C and 30C of the leading nodal ring 30 on the base end side and the tongue pieces 30B and 30B of the trailing nodal ring 30 on the distal end side overlap each other at the upper and lower positions or at the left and right positions, and are rotatably coupled together by coupling pins 36. Accordingly, the respective nodal rings 30 are alternately coupled together at the upper and lower positions and at the left and right positions.

Meanwhile, the distal end ring 32 that is the leading nodal ring 30 of the nodal ring group 14A, as illustrated in FIG. 2, has a ring-shaped outer peripheral part 80, and tongue pieces 82 and 82 are provided on both the left and right sides of the outer peripheral part 80 on the base end side. The tongue pieces 82 and 82 of the distal end ring 32 are rotatably coupled to the tongue pieces 30B and 30B of one trailing nodal ring 30 on the distal end side by the coupling pins 36.

Additionally, the distal end side of the outer peripheral part 80 is fixed to a base end of a main body 13A of the rigid distal end part 13.

In addition, the main body 13A of the rigid distal end part 13 is formed of metallic materials, such as stainless steel, and although the detailed description of the main body 13A is omitted, an image pick-up optical system 100 and a solid-state image pick-up element 102 that constitute the image pick-up device for picking up the image of a part to be observed, the illumination light emitting part (not illustrated) that radiates the illumination light, which is transmitted through the light guide 104 from the light source device, to a part to be observed, a forceps pipe 108 to which a forceps tube 106 that communicates with the forceps insertion part 16 of FIG. 1, is connected, and the like are assembled into the main body 13A. A signal cable 110 connected to the image pick-up device of the rigid distal end part 13, the light guide 104 connected to the illumination light emitting part, the forceps tube 106 connected to the forceps pipe 108, an air/water supply tube, and the like are inserted through and arranged inside the nodal rings 30 of the bending part 14 and inside the flexible tube part 15.

The base end ring 34 that is the trailing nodal ring 30 of the nodal ring group 14A, as illustrated in FIG. 3, has a ring-shaped outer peripheral part 50, and tongue pieces 52 and 52 which are provided on both the left and right sides of the outer peripheral part 50 on the distal end side. The tongue pieces 52 and 52 of the base end ring 34 are rotatably coupled to the tongue pieces 30C and 30C of one leading nodal ring 30 on the base end side by the coupling pins 36.

Additionally, the base end side of the outer peripheral part 50 is fixed to a distal end of a spiral pipe 15B that is covered with an sheath 15A of the flexible tube part 15 via an annular coupling ring 38.

The bending part 14 configured as described above is vertically and horizontally bent in a manner like the push/pull operation of an operating wire 40 passing through the respective nodal rings 30 of the nodal ring group 14A.

Four operating wires that pass through the respective upper and lower and left and right positions of the four directions inside the respective nodal rings 30 are arranged as the operating wire 40, and each operating wire 40 is inserted through and guided by through-holes 36A of wire guide portions that are formed integrally with the above-described coupling pins 36 coupling the nodal rings 30 and that protrude to the insides of the nodal rings 30.

A distal end part of each operating wire 40 is held by sleeves 84 provided at the respective upper and lower and left and right positions of the outer peripheral part 80 of the distal end ring 32 as illustrated in FIG. 2, and is fixed to the distal end ring 32.

Additionally, each operating wire 40, as illustrated in FIG. 3, is inserted through an inner hole 42A of a wire guide pipe 42 and is inserted through and arranged inside the flexible tube part 15, and a base end part of each operating wire 40 is connected to the bending operation mechanism in the operating part 11.

The wire guide pipe 42 is constituted of, for example, a closely wound coil, and four wire guide pipes passing through the respective upper and lower and left and right positions inside the flexible tube part 15 are arranged corresponding to the four operating wires 40. Distal end parts of the respective wide guide pipes 42 are held by sleeves 60 provided at the respective upper and lower and left and right positions of the outer peripheral part 50 of the base end ring 34 as illustrated in FIG. 3, and are fixed to the base end ring 34. A base end part of each wire guide pipe 42 is fixed to the inside of the operating part 11 or a base end part of the flexible tube part 15.

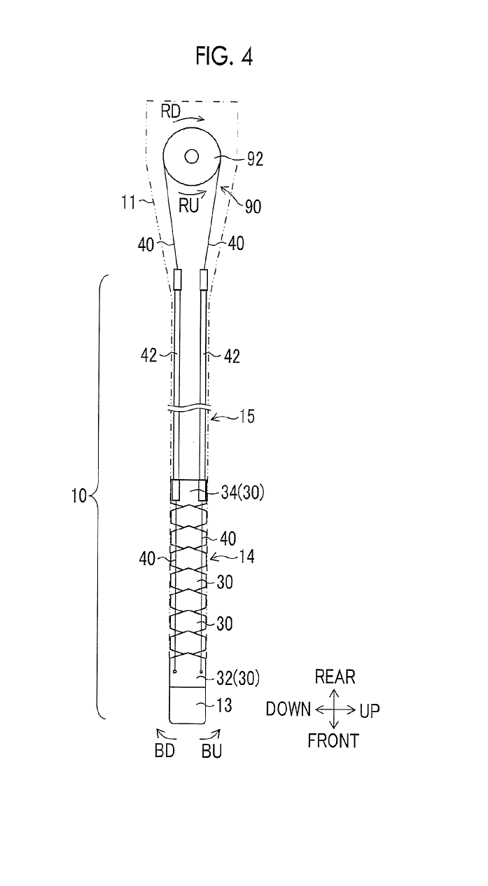

FIG. 4 is a view conceptually illustrating the bending operation mechanism of the bending part 14 and the operating part 11, and illustrates only the structure in which the bending part 14 is bent only in an upward-downward direction (an upward-downward direction on the plane of the drawing).

In the bending part 14, as illustrated in FIG. 4, the plurality of nodal rings 30 are coupled in series as described above, and the nodal rings 30 adjacent to each other are coupled together so as to be relatively rotatable around axes (coupling pins 36) along a leftward-rightward direction (a direction perpendicular to the plane of the drawing). In addition, in the respective nodal rings 30 of FIG. 4, two nodal rings coupled together so as to be rotatable around the axes in the upward-downward direction (the upward-downward direction on the plane of the drawing) are expressed as one nodal ring in FIGS. 2 and 3.

The distal end ring 32 that is the leading nodal ring 30 among the nodal rings 30 is fixed to the rigid distal end part 13, and the base end ring 34 that is the trailing nodal ring 30 is fixed to the flexible tube part 15. Accordingly, the entire bending part 14 is configured so as to be bendable in the upward-downward direction.

Additionally, distal end parts of a pair of operating wires 40 and 40 are respectively fixed to inner upper and lower parts of the distal end ring 32, and distal end parts of a pair of the wire guide pipes 42 and 42 through which the operating wires 40 are respectively inserted are respectively fixed to inner upper and lower parts of the base end ring 34.

The respective operating wires 40 are arranged through the inner upper and lower parts of the respective nodal rings 30 and the respective wire guide pipes 42, and the base end parts thereof are connected to the bending operation mechanism 90 within the operating part 11. That is, the base end parts of the respective operating wires 40 are wound around and fixed to a pulley 92 that constitutes the bending operation mechanism 90.

According to the above, if the pulley 92 is rotated by a surgeon's rotational operation of the upward-downward angle knob 22 (refer to FIG. 1) of the operating part 11 and is rotated in a direction RU in the drawing, the bending part 14 is bent upward (an arrow direction BU in the drawing). On the contrary, if the pulley 92 is rotated in a direction RD in the drawing, the bending part 14 is bent downward (arrow direction BD in the drawing).

In addition, as for the bending of the bending part 14 in the leftward-rightward direction, similarly, a pair of operating wires 40 and a pair of wire guide pipes 42 are arranged at inner left and right parts of the bending part 14 and the flexible tube part 15, and base ends of the operating wires 40 are wound around and fixed to a pulley (not illustrated) that is rotated during interlocking with the rotational operation of the leftward-rightward angle knob 23 (refer to FIG. 1) of the operating part 11. Accordingly, the bending part 14 is bent in the leftward-rightward direction by a surgeon's rotational operation of the leftward-rightward angle knob 23.

Next, a fixing structure of the distal end parts of the operating wires 40 and the wire guide pipes 42 will be described. As illustrated in FIG. 2, the operating wires 40 are fixed to the leading distal end ring 32 of the nodal ring group 14A in the bending part 14 by the sleeves 84, and as illustrated in FIG. 3, the wire guide pipes 42 are fixed to the trailing base end ring 34 of the nodal ring group 14A by the sleeves 60.

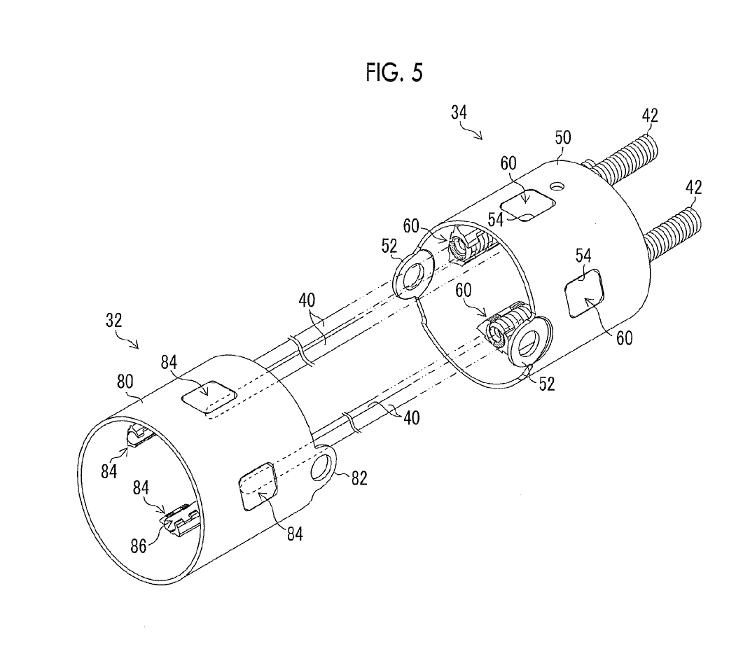

FIG. 5 is a perspective view illustrating only the distal end ring 32, the base end ring 34, the operating wires 40, and the wire guide pipes 42 in the bending part 14.

As illustrated in FIG. 5, the sleeves 84 are fixed to the inside of the outer peripheral part 80 of the distal end ring 32 at four places including upper and lower and left and right locations, and the distal end parts of the operating wires 40 are held by the sleeves 84.

Meanwhile, the sleeves 60 are fixed to the inside of the outer peripheral part 50 of the base end ring 34 at four places including lower and upper and left and right locations, and the distal end parts of the wire guide pipes 42 are held by the sleeves 60.

Here, all of the four sleeves 84 fixed to the outer peripheral part 80 of the distal end ring 32 and the four sleeves 60 fixed to the outer peripheral part 50 of the base end ring 34 hold the distal end parts of the operating wires 40 or the wire guide pipes 42 by means of the same configuration and the same method, and are fixed to the outer peripheral part 80 or the outer peripheral part 50.

Thus, only a configuration regarding a sleeve 60 fixed to an inner upper part of the outer peripheral part 50 of the base end ring 34 will be described below, and the other sleeves 60 and sleeves 84 are considered to be the same as the sleeve 60.

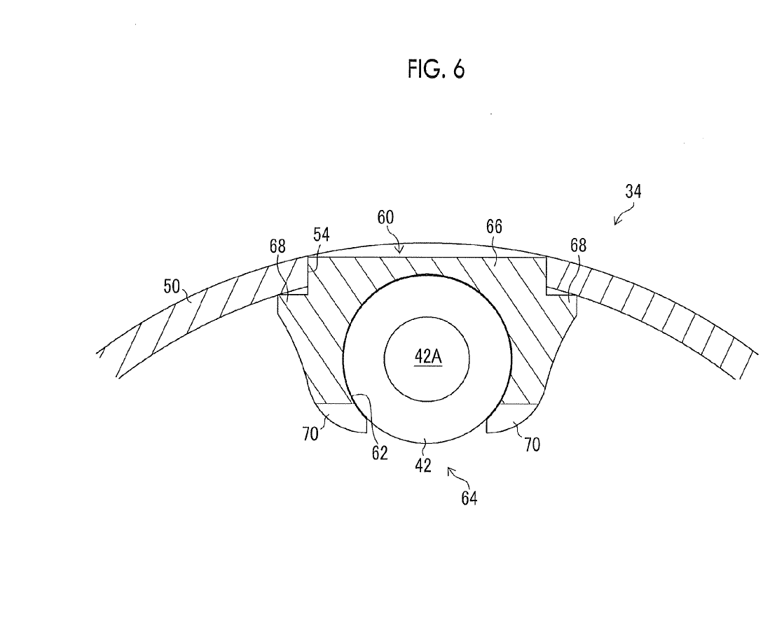

FIG. 6 is a cross-sectional view of the sleeve 60 provided in the inner upper part of the outer peripheral part 50 of the base end ring 34 that is cut in a plane orthogonal to a longitudinal axis of the insertion part 10, that is, a central axis of the outer peripheral part 50.

As illustrated in FIGS. 5 and 6, if a direction parallel to the longitudinal axis of the insertion part 10 is made to be an axial direction of the sleeve 60, the sleeve 60 is formed in a tubular shape of which the inside is hollow along the axial direction, and has a through-hole 62 that penetrates in the axial direction.

The internal diameter of the through-hole 62 is substantially the same as the external diameter of the wire guide pipe 42, and the distal end part of the wire guide pipe 42 is fitted into the through-hole 62 and thereby the sleeve 60 is externally fitted to the distal end part of the wire guide pipe 42 so as to hold the wire guide pipe 42. In addition, illustration of the wire guide pipe 42 in FIG. 6 is simplified.

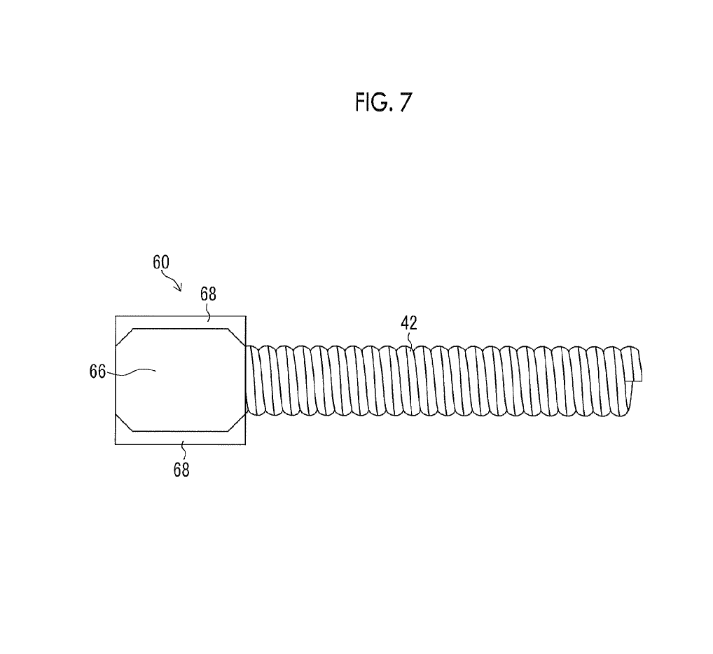

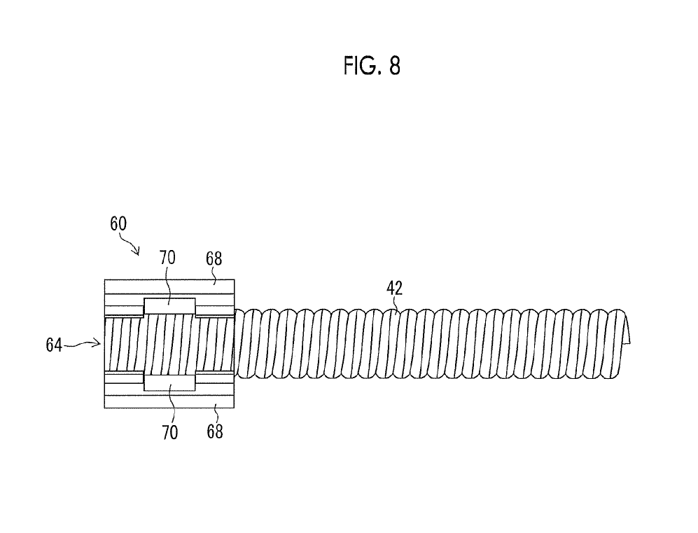

Additionally, each sleeve 60 is configured separately from the base end ring 34. FIG. 7 and FIG. 8 are views illustrating only the sleeve 60 and the wire guide pipe 42, and FIG. 7 is a view illustrating the sleeve 60 from a radial outer side with respect to the longitudinal axis of the insertion part 10, and FIG. 8 is a view illustrating the sleeve 60 from a radial inner side.

As illustrated in FIGS. 6 and 7, a projection 66 that protrudes in a rectangular shape is provided on an outer surface of the sleeve 60, that is, a surface opposite to a slit 64 to be described below, as a fixing part. Additionally, the sleeve 60 is formed with a vane 68 that protrudes in a circumferential direction of the outer peripheral part 50 of the base end ring 34.

Meanwhile, as illustrated in FIGS. 5 and 6, holes 54 having a shape (rectangular shape) corresponding to the projections 66 of the sleeves 60 are provided as fixed parts at four positions of the upper and lower and left and right locations in the outer peripheral part 50 of the base end ring 34.

Therefore, the sleeves 60 are fixed to the inside of the outer peripheral part 50 of the base end ring 34 in a state where the sleeves 60 are aligned with the base end ring 34 at predetermined positions by fitting the projections 66 that are the fixing parts of the sleeves 60 into the holes 54 of the outer peripheral part 50 that are the fixed parts of the base end ring 34.

Additionally, by making the vanes 68 of the sleeves 60 abut against an inner surface of the outer peripheral part 50 of the base end ring 34, the vanes 68 regulate the amount of insertion of the projections 66 into the holes 54 as position regulating parts, and outer surfaces of the projections 66 are fixed at locations with a small height difference such that bulging does not occur with respect to an outer peripheral surface of the outer peripheral part 50 of the base end ring 34. Additionally, the sleeves 60 are fixed in a stable state without shaking or the like with respect to the outer peripheral part 50.

Laser welding is performed for the fixation between the sleeves 60 and the outer peripheral part 50 of the base end ring 34, and the laser welding is performed, for example, by radiating laser light to contact portions between the projections 66 of the sleeves 60 and the holes 54 of the outer peripheral part 50 of the base end ring 34.

Meanwhile, as illustrated in FIGS. 6 and 8, the slit 64, which is cut out along the axial direction of the sleeve 60 and allows the inside (through-hole 62) to the communicate with the outside, is formed at a partition wall that becomes the inside of the sleeve 60, that is, the center side of the insertion part 10 (base end ring 34). The slit 64 is formed as a result of reducing a partition wall portion, which is located closer to an inner side than the wire guide pipe 42 fixed to the through-hole 62 of the sleeve 60, as much as possible, in order to make the internal space in the insertion part 10 as wide as possible.

In this case, if the inner partition wall portion of the sleeve 60 is excessively reduced, the maximum internal diameter of a hollow tubular inner part (through-hole 62) of the sleeve 60 is not maintained, and the sleeve 60 cannot hold the wire guide pipe 42. Therefore, the inner partition wall portion of the sleeve 60 is cut out to such a degree that the slit 64 can maintain the maximum internal diameter of the hollow tubular inner part of the sleeve 60.

Additionally, if the opening width of the slit 64 in a direction perpendicular to the axial direction of the sleeve 60 is excessively wide, the wire guide pipe 42 is easily separated via the slit 64. Therefore, the opening width of the slit 64 is made smaller than at least the external diameter of the wire guide pipe 42.

Additionally, as illustrated in FIGS. 6 and 8, a laser welded part 70 consisting of a cutout part that is cut out in a radial direction perpendicular to the axial direction is formed in the vicinity of the center of the sleeve 60 in the axial direction (the direction of the longitudinal axis of the insertion part 10) in an outer wall that defines the slit 64. The laser welded part 70 becomes a portion that performs laser welding as a contact portion between the sleeve 60 and the wire guide pipe 42. If the laser welding is performed, the welded portion bulges. However, the laser welded part 70 is dented in advance, thereby preventing the portion, which has bulged due to the laser welding, from protruding further than a peripheral portion of the bulging portion.

In addition, the cutout part that is the laser welded part 70 may be formed in regions other than the vicinity of the center of the sleeve 60 in the axial direction within a range of the outer wall that defines the slit 64.

To describe a work sequence in which the distal end parts of the wire guide pipes 42 are fixed to the outer peripheral part 50 of the base end ring 34 via the above sleeves 60, first, the through-holes 62 of the sleeves 60 that are not fixed to the outer peripheral part 50 of the base end ring 34 are externally fitted to the distal end parts of the wire guide pipes 42, and laser light is radiated to the contact portions between the sleeves 60 and the wire guide pipes 42 in the laser welded parts 70, thereby fixing the sleeves 60 and the wire guide pipes 42 by means of laser welding.

Subsequently, the projections 66 of the sleeves 60 fixed to the distal end parts of the wire guide pipes 42 are fitted into the holes 54 from the inside of the outer peripheral part 50 of the base end ring 34, and the vanes 68 of the sleeves 60 are made to abut against the inner peripheral surface of the outer peripheral part 50 and are fixed in that state using a jig or the like. Then, laser light is radiated to the contact portions between the projections 66 (side wall surface) of the sleeves 60 and the holes 54 (inner wall surface) of the outer peripheral part 50 from an outer peripheral side of the outer peripheral part 50, and the sleeves 60 and the outer peripheral part 50 are fixed to each other by the laser welding.

Accordingly, the distal end parts of the wire guide pipes 42 are fixed to the base end ring 34 via the sleeves 60.

In addition, as described above, the sleeves 84 that are fixed to the outer peripheral part 80 of the distal end ring 32 and hold the distal end parts of the operating wires 40 are configured similarly to the sleeves 60, and have slits 86 as illustrated in FIG. 5. The opening width of the slits 86 is smaller than at least the external diameter of the operating wires 40.

In the above embodiment, the sleeves 60 are provided separately from the outer peripheral part 50 of the base end ring 34. However, the sleeve 60 may be integrally formed on the outer peripheral part 50. The same applies to the sleeves 84.

Additionally, in the above embodiment, the sleeves 60 and the wire guide pipes 42 are fixed to each other by the laser welding and the sleeves 60 and the outer peripheral part 50 of the base end ring 34 are fixed to each other by the laser welding. However, these may be fixed using welding other than the laser welding or may be fixed using an adhesive. The same applies to the sleeves 84.

Additionally, an aspect in which the distal end parts of the wire guide pipes 42 are fixed to the base end ring 34 in the base end part of the bending part 14 is illustrated in the above embodiment. However, the distal end parts of the wire guide pipes 42 may be fixed to a ring member or the like in the distal end part of the flexible tube part 15. In that case, the same configuration as that of the above embodiment can also be applied. Similarly, an aspect in which the distal end parts of the operating wires 40 are fixed to the distal end ring 32 in the distal end part of the bending part 14 is illustrated in the above embodiment. However, the distal end parts of the operating wires 40 may be fixed to a ring member or the like in the rigid distal end part 13. In that case, the same configuration as that of the above embodiment can be applied.

Additionally, the configuration for fixing the sleeves 60 to the outer peripheral part 50 is not limited to that illustrated in the above embodiment.

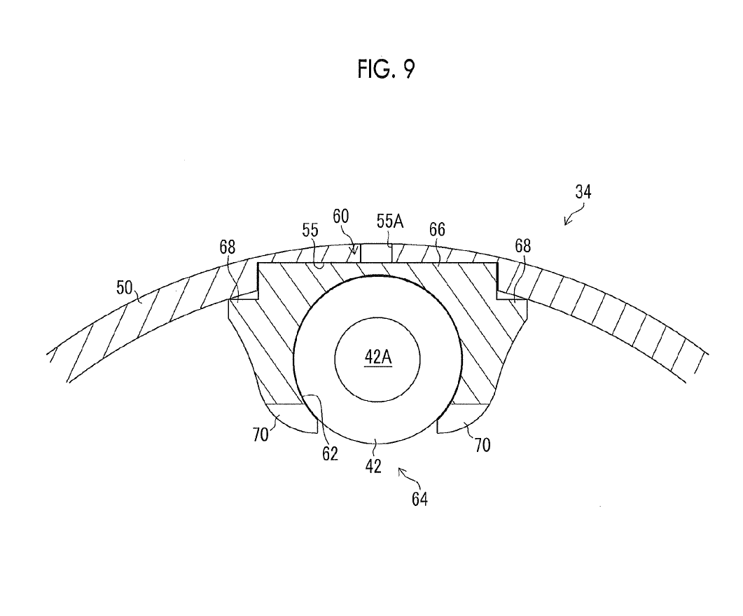

For example, a groove 55 serving as a fixed part is formed on the inner surface side of the outer peripheral part 50 as illustrated in FIG. 9, instead of providing the hole 54 that is the fixed part in the outer peripheral part 50 of the base end ring 34 as illustrated in FIGS. 5 and 6. Meanwhile, the projection 66 that is the fixing part of the sleeve 60 has a shape such that the projection is engageable with the groove 55, and has an amount of protrusion such that the vane 68 is able to abut against the inner surface of the outer peripheral part 50. Accordingly, the sleeve 60 can be fixed to the inside of the outer peripheral part 50 in a state where the vane 68 of the sleeve 60 is made to abut against the inner surface of the base end ring 34 by fitting the projection 66 of the sleeve 60 into the groove 55 of the outer peripheral part 50 of the base end ring 34 and performing welding or bonding therebetween as shown in FIG. 9.

Additionally, when the sleeve 60 is laser-welded to the outer peripheral part 50, a through-hole 55A for radiating laser light to the contact portion between the sleeve 60 and the outer peripheral part 50 may be formed as shown in FIG. 9 in the outer peripheral part 50 from the outside of the outer peripheral part 50. In FIG. 9, the through-hole 55A is formed in a columnar shape or a prismatic shape substantially at a center position of the range of the groove 55. However, the disclosure is not limited to this, and an aspect may be adopted in which an arbitrary number of through-holes 55A are formed at arbitrary positions of the range of the groove 55. Additionally, since a gap is hardly generated between the inner peripheral surface of the outer peripheral part 50 and a facing surface of the vane 68 that faces the inner peripheral surface thereof, or as the facing surface is formed in a curved surface shape such that the facing surface comes into surface contact with the inner peripheral surface of the outer peripheral part 50, it is also possible to form the through-hole 55A within the range of the outer peripheral part 50 that faces the vane 68. Moreover, the through-hole 55A can also be formed as an elongated hole that is long in one direction.

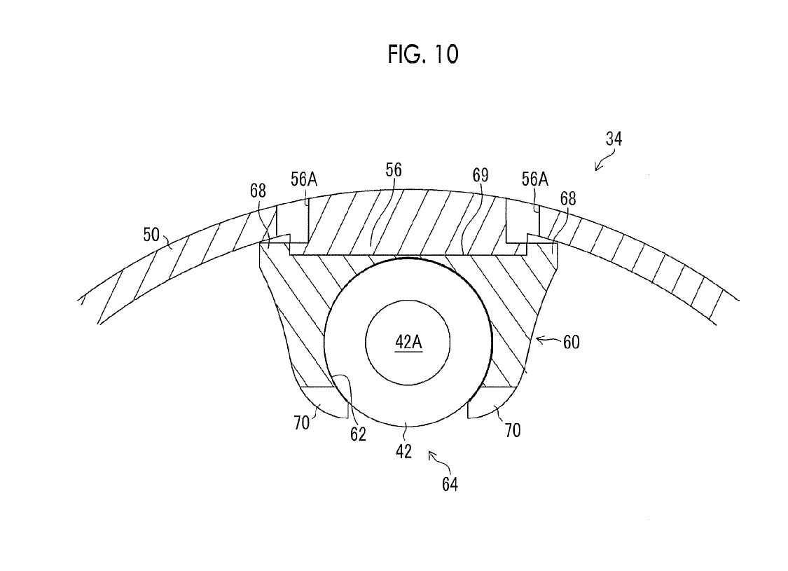

Additionally, as illustrated in FIG. 10, contrary to the aspect of FIG. 9, a projection 56 serving as a fixing part is provided on the inner surface side of the outer peripheral part 50 of the base end ring 34, and a groove 69 serving as a fixed part is provided in the surface of the sleeve 60 opposite to the slit 64. The projection 56 of the base end ring 34 has a shape such that the projection is engageable with the groove 69 of the sleeve 60, and has an amount of protrusion such that the vane 68 is able to abut against the inner surface of the outer peripheral part 50. Accordingly, the sleeve 60 can be fixed to the inside of the outer peripheral part 50 in a state where the vane 68 of the sleeve 60 is made to abut against the inner surface of the base end ring 34 by fitting the projection 56 of the outer peripheral part 50 of the base end ring 34 into the groove 69 of the sleeve 60 and performing welding or bonding therebetween as shown in FIG. 10.

Additionally, when the sleeve 60 is laser-welded to the outer peripheral part 50, a through-hole 56A for radiating laser light to the contact portion between the sleeve 60 and the outer peripheral part 50 may be formed as shown in FIG. 10 in the outer peripheral part 50 from the outside of the outer peripheral part 50. In FIG. 10, the through-hole 56A is formed in a columnar shape or a prismatic shape at the position of a thin peripheral edge in the range of the projection 56. However, the disclosure is not limited to this, and an aspect may be adopted in which an arbitrary number of through-holes 56A are formed at arbitrary positions within the range of the projection 56. Additionally, since a gap is hardly generated between the inner peripheral surface of the outer peripheral part 50 and the facing surface of the vane 68 that faces the inner peripheral surface thereof, or as the facing surface is formed in a curved surface shape such that the facing surface comes into surface contact with the inner peripheral surface of the outer peripheral part 50, it is also possible to form the through-hole 56A within the range of the outer peripheral part 50 that faces the vane 68. Moreover, the through-hole 56A can also be formed as an elongated hole that is long in one direction.

Additionally, a case which the distal end parts of the operating wires 40 and the wire guide pipes 42 relating to the bending operation of the bending part 14 are fixed to the ring member of the distal end ring 32, the base end ring 34, or the like by the sleeves 60 and 84 has been described in the above embodiment. However, the disclosure is not limited to the operating wires 40 and the wire guide pipes 42. The same configuration and fixing method as those of the above embodiment can also be applied to a case where a portion of an arbitrary insertion member inserted through the insertion part 10 of the endoscope 2 is externally fitted and fixed to a tubular sleeve. That is, a configuration in which a slit having an opening width smaller than the external diameter of the insertion member is formed in the sleeve can be adopted similar to the above embodiment. Additionally, a method of fixing an insertion member including a step of externally fitting the sleeve to a portion of the insertion member, and a step of fixing the sleeve to the insertion member by performing laser welding via the slit may be adopted. As an insertion member other than the above operating wires 40 or wire guide pipes 42, for example, an operating wire or the like for adjusting the hardness of the flexible tube part 15 of the insertion part 10 is used.

What is claimed is:

1. An endoscope comprising:
an insertion part in which a rigid distal end part, a bending part, and a flexible tube part are provided in order from a distal end side to a base end side;
an operating part having a bending operation mechanism that performs bending of the bending part, the operating part being provided on the base end side of the insertion part;
an operating wire having a distal end part and a base end part, the distal end part being fixed to a distal end part of the bending part or the rigid distal end part, the base end part being connected to the bending operation mechanism, the operating wire being configured to bend the bending part by the bending operation mechanism being operated;
a wire guide pipe through which the operating wire is inserted, having a distal end part and a base end part, the distal end part being fixed to a base end part of the bending part or a distal end part of the flexible tube part, the base end part being fixed to an inside of the operating part or a base end part of the flexible tube part; and
a sleeve of which an inside is formed in a hollow tubular shape along an axial direction parallel to a longitudinal axis of the insertion part and which is externally fitted to and hold the distal end part of the operating wire or the wire guide pipe,
wherein the sleeve includes a slit that is formed by cutting out a partition wall of the insertion part on a center side along the axial direction in a section perpendicular to the axial direction, and that allows the inside and an outside of the sleeve to communicate with each other,
wherein the slit maintains a maximum internal diameter of the inside formed in the hollow tubular shape, and
wherein an opening width of the slit in a circumferential direction perpendicular to the axial direction is made to be smaller than an external diameter of the distal end part of the operating wire inserted through the sleeve or the wire guide pipe.

2. The endoscope according to claim 1,
wherein the sleeve has a laser welded part including a cutout part formed by partially cutting out an outer wall defining the slit in a radial direction perpendicular to the axial direction.

3. The endoscope according to claim 1, further comprising:
a ring member that constitutes a part of an outer peripheral part of the insertion part,
wherein the sleeve is constituted separately from the ring member, and is fixed to an inside of the ring member.

4. The endoscope according to claim 2, further comprising:
a ring member that constitutes a part of an outer peripheral part of the insertion part,
wherein the sleeve is configured separately from the ring member, and is fixed to an inside of the ring member.

5. The endoscope according to claim 3,
wherein the sleeve is fixed to the inside of the ring member by laser welding.

6. The endoscope according to claim 4,
wherein the sleeve is fixed to the inside of the ring member by laser welding.

7. The endoscope according to claim 3,
wherein either one of the sleeve and the ring member has a fixed part, and the other one of the sleeve and the ring member has a fixing part that is engageable with the fixed part, and
wherein the sleeve is fixed in a state where the sleeve is aligned with the ring member by engaging the fixed part with the fixing part.

8. The endoscope according to claim 4,
wherein either one of the sleeve and the ring member has a fixed part, and the other one of the sleeve and the ring member has a fixing part that is engageable with the fixed part, and
wherein the sleeve is fixed in a state where the sleeve is aligned with the ring member by engaging the fixed part with the fixing part.

9. The endoscope according to claim 5,
wherein either one of the sleeve and the ring member has a fixed part, and the other one of the sleeve and the ring member has a fixing part that is engageable with the fixed part, and
wherein the sleeve is fixed in a state where the sleeve is aligned with the ring member by engaging the fixed part with the fixing part.

10. The endoscope according to claim 6,
wherein either one of the sleeve and the ring member has a fixed part, and the other one of the sleeve and the ring member has a fixing part that is engageable with the fixed part, and
wherein the sleeve is fixed in a state where the sleeve is aligned with the ring member by engaging the fixed part with the fixing part.

11. The endoscope according to claim 7,
wherein the fixing part is constituted by a projection provided on a surface of the sleeve opposite to the slit,
wherein the fixed part is constituted by a hole or a groove that is provided in the ring member and has a shape corresponding to the projection, and
wherein the sleeve is fixed in a state where the sleeve is aligned with the ring member by fitting the projection into the hole or the groove.

12. The endoscope according to claim 8,
wherein the fixing part is constituted by a projection provided on the surface of the sleeve opposite to the slit,
wherein the fixed part is constituted by a hole or a groove that is provided in the ring member and has a shape corresponding to the projection, and
wherein the sleeve is fixed in a state where the sleeve is aligned with the ring member by fitting the projection into the hole or the groove.

13. The endoscope according to claim 9,
wherein the fixing part is constituted by a projection provided on the surface of the sleeve opposite to the slit,
wherein the fixed part is constituted by a hole or a groove that is provided in the ring member and has a shape corresponding to the projection, and
wherein the sleeve is fixed in a state where the sleeve is aligned with the ring member by fitting the projection into the hole or the groove.

14. The endoscope according to claim 10,
wherein the fixing part is constituted by a projection provided on the surface of the sleeve opposite to the slit,
wherein the fixed part is constituted by a hole or a groove that is provided in the ring member and has a shape corresponding to the projection, and
wherein the sleeve is fixed in a state where the sleeve is aligned with the ring member by fitting the projection into the hole or the groove.

15. The endoscope according to claim 11,
wherein the fixing part is constituted by a projection provided on the surface of the sleeve opposite to the slit,
wherein the fixed part is constituted by a hole or a groove that is provided in the ring member and has a shape corresponding to the projection, and
wherein the sleeve is fixed in a state where the sleeve is aligned with the ring member by fitting the projection into the hole or the groove.

16. The endoscope according to claim 11,
wherein the sleeve has a position regulating part that abuts against an inner surface of the ring member when the projection is fitted into the hole or the groove.

17. The endoscope according to claim 12,
wherein the sleeve has a position regulating part that abuts against an inner surface of the ring member when the projection is fitted into the hole or the groove.

18. The endoscope according to claim 13,
wherein the sleeve has a position regulating part that abuts against an inner surface of the ring member when the projection is fitted into the hole or the groove.

19. A part fixing structure for fixing an insertion member, which is inserted through an insertion part of an endoscope, comprising:
a sleeve of which an inside is formed in a hollow tubular shape along an axial direction parallel to a longitudinal axis of the insertion part and which is externally fitted to and hold a part of the insertion member,
wherein the sleeve includes a slit that is formed by cutting out a partition wall of the insertion part on a center side along the axial direction in a section perpendicular to the axial direction, and that allows the inside and an outside of the sleeve to communicate with each other,
wherein the slit maintains a maximum internal diameter of the inside formed in the hollow tubular shape, and
wherein an opening width of the slit in a direction perpendicular to the axial direction is made to be smaller than an external diameter of the insertion member.

20. A part fixing method for fixing an insertion member inserted through an insertion part of an endoscope, comprising:
a step of having a sleeve to be externally fitted to a part of the insertion member, of which sleeve an inside is formed in a hollow tubular shape along an axial direction parallel to a longitudinal axis of the insertion part and which includes a slit which is formed by cutting out a partition wall of the insertion part on a center side along the axial direction in a section perpendicular to the axial direction, the slit allowing the inside and the outside of the sleeve to communicate with each other, wherein the slit maintains a maximum internal diameter of the inside formed in the hollow tubular shape, and an opening width of the slit in a direction perpendicular to the axial direction is made to be smaller than an external diameter of the insertion member; and
a step of fixing the insertion member to the sleeve by performing laser welding via the slit.
